(19) United States
(12) Patent Application Publication
(10) Pub. No.: US 2025/0145964 A1
(43) Pub. Date: May 8, 2025

(54) COMPOSITIONS AND METHODS FOR USING INDIVIDUALIZED GENOME ASSEMBLIES AND INDUCED PLURIPOTENT STEM CELL LINES OF NONHUMAN PRIMATES FOR PRE-CLINICAL EVALUATION

(71) Applicants: EXIR, INC., Sunnyvale, CA (US); EXIR LLC, Woodland, CA (US)

(72) Inventor: Morteza Roodgar, Menlo Park, CA (US)

(21) Appl. No.: 18/707,061

(22) PCT Filed: Oct. 20, 2022
(86) PCT No.: PCT/US2022/078407

§ 371 Date: May 2, 2024

Related U.S. Application Data
(60) Provisional application No. 63/257,997, filed on Oct. 20, 2021.

(51) Int. Cl. C12N 5/074; C12N 15/10

(52) U.S. Cl. CPC C12N 5/0696; C12N 15/1093

(57) ABSTRACT

Described herein are methods for pre-clinical drug evaluation including using an individualized genome assembly for a test subject; and induced pluripotent stem cells (iPSCs) derived from the test subject. Further, methods for preclinical testing of human therapies include: obtaining tissue or blood from at least one individual of a population comprising a single species of nonhuman primate; isolating PBMCs from the at least one individual deriving iPSCs from the PBMCs from the at least one individual; performing one or more of: in vitro, in vivo, or genetic testing on the at least one individual. Compositions for deriving iPSCs from nonhuman primates are also described herein.

### CROSS-REFERENCE TO RELATED APPLICATIONS

This application claims the priority benefit of U.S. Provisional Patent Application Ser. 63/257,997, filed Oct. 20, 2021, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates to bioinformatics, potency, functionality, and toxicology of pharmaceuticals and related chemicals for preclinical testing. In addition, this disclosure relates to compositions and methods for using individualized genome assemblies and induced pluripotent stem cell lines of nonhuman primates for pre-clinical evaluation.

### BACKGROUND

In the pharmaceutical industry, pre-clinical research helps a manufacturer to determine if a drug candidate is safe to be tested on humans. Conventionally, pre-clinical testing relies heavily on animal testing. Typically, FDA policy requires that a drug candidate be tested in a rodent species and a non-rodent species. While there is no requirement that the non-rodent species be a primate, nonhuman primates (NHP) are often used in pre-clinical testing because of their close anatomic and physiologic similarity to humans. The primate subjects are exposed to the drug candidate to help determine the candidate's safety profile for humans. Use of nonhuman primates is extremely expensive. In some cases, a single animal can cost tens of thousands of dollars only to be sacrificed during the testing. Additionally, there is a growing distaste for animal testing in general and NHP testing in particular.

Accordingly, there exists a great need for testing approaches that minimize the number of animals that must be sacrificed during pre-clinical testing.

### SUMMARY

In some aspects, the techniques described herein relate to a method for deriving individual pluripotent stem cells (iPSCs) from individuals within a population of nonhuman primates (NHP) including: obtaining peripheral blood mononuclear cells (PBMCs) of at least one individual of a population including a single species of nonhuman primate; culturing the PBMCs to expand blood progenitor cells in a hematopoietic stem cell (HSC) expansion medium for a predetermined time period; transfecting the cultured PBMCs with a combination of transcription factors so that the cells are induced to overexpress the transcription factors, such that the transfected cultured PBMCs are reprogrammed into iPSCs; no more than one day post-transfection, transferring the transfected cells into a container including a plurality of feeder cells; no more than one day after said transferring, transferring the cells to a second container; on selected days after transfer to the second container, performing at least one of: adding medium to the cells; changing the medium in the cells; and adding one or more cell growth factors to the cells; until a first cell colony appears; passaging the cells by placing each colony in a third container coated with an extracellular matrix; and expanding the first cell colony for use in one or more of: an in vitro test, an in vivo test, or creation of a genome library.

In some aspects, the techniques described herein relate to a method, including the expanding includes washing the iPSCs with phosphate-buffered saline (PBS) and incubating the iPSCs with a cell detachment solution.

In some aspects, the techniques described herein relate to a method, including the incubating occurs at about 37 degrees Celcius for a predetermined time period.

In some aspects, the techniques described herein relate to a method, including the expanding includes aspirating and dissociating the iPSCs into a single cell suspension.

In some aspects, the techniques described herein relate to a method, further including differentiating the iPSCs into main lineages, the main lineages including at least one of: ectoderm, mesoderm, and endoderm.

In some aspects, the techniques described herein relate to a method, further including differentiating the main lineages into final tissues.

In some aspects, the techniques described herein relate to a method, further including generating an individualized genome assembly of the at least one individual.

In some aspects, the techniques described herein relate to a method, further including extracting a quantity of high-molecular weight (HMW) DNA from the obtained PBMCs by means of a process for purification of archival quality DNA.

In some aspects, the techniques described herein relate to a method, further including preparing a genomic library from the extracted HMW DNA; sequencing long-read fragments of HMW DNA; and assembling and annotating the long-read fragments to recreate the genome of the at least one individual from which the PBMCs were obtained.

In some aspects, the techniques described herein relate to a method, including the transcription factors are selected from a group consisting of: c-myc, Klf4, Sox2, Oct3/4, Klf2, Nanog, Tfcp2L1, and Stat3.

In some aspects, the techniques described herein relate to a method, including the plurality of feeder cells are mouse embryonic fibroblast (MEF) cells or SNL feeder cells.

In some aspects, the techniques described herein relate to a method, including the transferring the transfected cells into a container including the plurality of feeder cells further includes adding a tankyrase 1/2 inhibitor in a range of about 1 μM to about 3 μM.

In some aspects, the techniques described herein relate to a method, including a ratio of transfected cells to the plurality of feeder cells is about 1:3 to about 1:7.

In some aspects, the techniques described herein relate to a method, including the transfecting is using a Sendai virus vector.

In some aspects, the techniques described herein relate to a method, including the one or more cell growth factors include basic fibroblast growth factor.

In some aspects, the techniques described herein relate to a method, including the passaging the cells by placing each colony in the third container coated with the extracellular matrix includes adding a serine/threonine kinase inhibitor to a medium in the third container.

In some aspects, the techniques described herein relate to a method for pre-clinical drug evaluation including: an individualized genome assembly for a test subject; and induced pluripotent stem cells (iPSCs) derived from the test subject.

In some aspects, the techniques described herein relate to a method, further including implanting cells or tissues derived from iPSCs of the test subject into the test subject to evaluate functionality of the implanted cells or tissues of the test subject.

In some aspects, the techniques described herein relate to a method, further including generating one or more panels; and predicting whether a particular community of animals is a fit for a drug experiment based on the one or more panels.

In some aspects, the techniques described herein relate to a method, further including generating a guide RNA or gene-editing tool using the iPSCs derived from the test subject; and performing in vivo testing of the guide RNA or gene-editing tool in the test subject.

In some aspects, the techniques described herein relate to a method for preclinical testing of human therapies including: obtaining tissue or blood from at least one individual of a population including a single species of nonhuman primate; isolating PBMCs from the at least one individual; deriving iPSCs from the PBMCs from the at least one individual; extracting DNA from the PBMCs from the at least one individual; preparing a DNA library using, at least in part, the extracted DNA; preparing a full genome assembly for the at least one individual; and performing computational analysis on the full genome assembly.

In some aspects, the techniques described herein relate to a method, including the computational analysis includes identifying one or both of: Major Histocompatibility Complex related genes or Minor Histocompatibility complex related genes.

In some aspects, the techniques described herein relate to a method, including the computational analysis includes identifying immune system-related genes.

In some aspects, the techniques described herein relate to a method, including the computational analysis includes identifying anti-drug antibody (ADA) response related genes.

In some aspects, the techniques described herein relate to a method, including the computational analysis includes performing preclinical genotyping.

In some aspects, the techniques described herein relate to a method, including the computational analysis includes identifying liver toxicity related genes.

In some aspects, the techniques described herein relate to a method, including the DNA is high molecular weight DNA.

In some aspects, the techniques described herein relate to a method, including the DNA is archival quality DNA.

In some aspects, the techniques described herein relate to a method, including the preparing the DNA library includes performing long read sequencing on the extracted DNA.

In some aspects, the techniques described herein relate to a method, including the preparing the full genome assembly includes recreating a genome of the at least on individual based on the prepared DNA library.

In some aspects, the techniques described herein relate to a method for preclinical testing of human therapies including: obtaining tissue or blood from at least one individual of a population including a single species of nonhuman primate; isolating PBMCs from the at least one individual; deriving iPSCs from the PBMCs from the at least one individual; performing in vitro testing on the derived iPSCs from the at least one individual; deriving in vitro testing results from the in vitro testing; and correlating the in vitro testing results with a genotype of the at least one individual.

In some aspects, the techniques described herein relate to a method, including the performing in vitro testing includes designing a guide RNA and transfecting the iPSCs in vitro with the guide RNA.

In some aspects, the techniques described herein relate to a method, including the deriving in vitro testing results includes running a panel to detect a gene editing efficiency of the guide RNA in vitro in the iPSCs.

In some aspects, the techniques described herein relate to a method for preclinical testing of human therapies including: obtaining tissue or blood from at least one individual of a population including a single species of nonhuman primate; isolating PBMCs from the at least one individual; deriving iPSCs from the PBMCs from the at least one individual; performing in vivo testing in the at least one individual; deriving in vivo testing results from the in vivo testing; and correlating in vivo testing results from the in vivo testing with a genotype of at least one individual.

In some aspects, the techniques described herein relate to a method, including the deriving the iPSCs includes: culturing the PBMCs to expand blood progenitor cells in a hematopoietic stem cell (HSC) expansion medium for a predetermined time period; transfecting the cultured PBMCs with a combination of transcription factors so that the cells are induced to overexpress the transcription factors, such that the transfected cultured cells are reprogrammed into iPSCs; no more than one day post-transfection, transferring the transfected cells into a container including a plurality of feeder cells; no more than one day after said transferring, transferring the cells to a second container; on selected days after transfer to the second container, performing at least one of: adding medium to the cells; changing the medium in the cells; and adding one or more cell growth factors to the cells; until a first cell colony appears; passaging the cells by placing each colony in a third container coated with an extracellular matrix; and expanding the first cell colony for use in one or more of: an in vitro test, an in vivo test, or creation of a genome library.

In some aspects, the techniques described herein relate to a method, including the performing in vivo testing includes autologously transplanting or injecting a subset of the iPSCs into the at least one individual.

In some aspects, the techniques described herein relate to a method, further including labeling the iPSCs before autologous transplantation or injection.

In some aspects, the techniques described herein relate to a method, including labeling includes fluorescent labeling.

In some aspects, the techniques described herein relate to a method, including the deriving the in vivo testing results includes running a panel to detect anti-drug antibodies.

In some aspects, the techniques described herein relate to a method, including the deriving the in vivo testing results includes running a panel to detect liver toxicity.

In some aspects, the techniques described herein relate to a method for preclinical testing of human therapies including: obtaining tissue or blood from at least one individual of a population including a single species of nonhuman primate; isolating PBMCs from the at least one individual; deriving iPSCs from the PBMCs from the at least one individual; performing in vitro testing on a subset of the derived iPSCs from the at least one individual; deriving in vitro testing results from the in vitro testing; autologously injecting or transplanting a second subset of the derived iPSCs from the at least one individual into the at least one individual; performing in vivo testing in the at least one individual; deriving in vivo testing results from the in vivo testing; and correlating the in vivo testing results with the in vitro testing results.

In some aspects, the techniques described herein relate to a method, including the deriving iPSCs includes: culturing the PBMCs to expand blood progenitor cells in a hematopoietic stem cell (HSC) expansion medium for a predetermined time period; transfecting the cultured PBMCs with a combination of transcription factors so that the cells are induced to overexpress the transcription factors, such that the transfected cultured cells are reprogrammed into iPSCs; no more than one day post-transfection, transferring the transfected cells into a container including a plurality of feeder cells; no more than one day after said transferring, transferring the cells to a second container; on selected days after transfer to the second container, performing at least one of: adding medium to the cells; changing the medium in the cells; and adding one or more cell growth factors to the cells; until a first cell colony appears; passaging the cells by placing each colony in a third container coated with an extracellular matrix; and expanding the first cell colony for use in one or more of: an in vitro test, an in vivo test, or creation of a genome library.

In some aspects, the techniques described herein relate to a method, including the performing in vitro testing includes designing a guide RNA and transfecting the subset of iPSCs in vitro with the guide RNA.

In some aspects, the techniques described herein relate to a method, including the deriving in vitro testing results includes running a panel to detect an in vitro gene editing efficiency of the guide RNA in the subset of iPSCs.

In some aspects, the techniques described herein relate to a method, further including transfecting iPSCs with the guide RNA for the autologous transplantion or injection.

In some aspects, the techniques described herein relate to a method, including the deriving the in vivo testing results includes running a panel to detect liver toxicity.

In some aspects, the techniques described herein relate to a method, including the deriving the in vivo testing results includes running a panel to detect an in vivo gene editing efficiency of the guide RNA in the second subset of iPSCs.

In some aspects, the techniques described herein relate to a method, including the correlating includes comparing the in vitro gene editing efficiency to the in vivo gene editing efficiency.

In some aspects, the techniques described herein relate to a method, including the performing in vitro testing includes treating the iPSCs in vitro with a therapy.

In some aspects, the techniques described herein relate to a method, including the deriving the in vitro testing results includes running a panel to detect an in vitro toxicity of the therapy.

In some aspects, the techniques described herein relate to a method, including the performing the in vivo testing results includes treating the at least one individual with the therapy.

In some aspects, the techniques described herein relate to a method, including the deriving the in vivo testing results includes running a panel to detect an in vivo toxicity of the therapy.

In some aspects, the techniques described herein relate to a method, including the correlating includes comparing the in vitro toxicity to the in vivo toxicity of the therapy.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 is a schematic showing an embodiment of a method for preclinical testing of human therapies using induced pluripotent stem cells (iPSCs) from nonhuman primates, genome assembly, and related genomic computations.

FIG. 2 is a schematic showing the differentiation of primate iPSCs to different tissues and cells to evaluate potency, functionality, and/or toxicity of a candidate therapy in vitro before evaluating that therapy in the same nonhuman primate from which the iPSCs were derived.

FIG. 3A is a flow chart showing an embodiment of a method for reprogramming nonhuman primate peripheral blood mononuclear cells (PBMCs) into iPSCs.

FIG. 3B is a schematic of an embodiment of a method for reprogramming nonhuman primate peripheral blood mononuclear cells (PBMCs) into iPSCs.

FIG. 4 is a schematic showing an embodiment of a method for in vitro testing of drugs on nonhuman primate iPSC-derived tissues and/or cells prior to potency, functionality, or safety evaluation of the therapies in vivo in the same nonhuman primate from which the iPSC cells and/or tissues were derived.

FIG. 5 shows a schematic of an embodiment of a method for in vitro testing of gene therapy products on iPSCs or iPSCs-derived cells to evaluate potency, functionality, and/or toxicity prior to testing in vivo in the same nonhuman primate from which the iPSC cells and/or tissues were derived.

FIG. 6A is a schematic showing an embodiment of a method of autologous transplantation of iPSCs or iPSC-derived cells and/or tissues into the same nonhuman primate from which the iPSCs were derived as a model for preclinical testing of cell and/or gene therapies in humans.

FIG. 6B shows labeling of iPSCs for further studies.

FIG. 6C shows gene editing in iPSC cells or tissues.

FIG. 7A is a 4× magnification of macaque iPSCs derived using conventional compositions and methods that were developed for human iPSCs.

FIG. 7B is a 10× magnification of macaque iPSCs derived using conventional compositions and methods that were developed for human iPSCs.

FIG. 7C is a 4× magnification of macaque iPSCs derived using the nonhuman primate iPSC compositions and methods described herein.

FIG. 7D is a 10× magnification of macaque iPSCs derived using the nonhuman primate iPSC compositions and methods described herein.

FIG. 7E is a graph showing the efficiency of iPSC derivation using conventional compositions and methods that were developed for human iPSCs versus using the nonhuman primate iPSC compositions and methods described herein.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated embodiment(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Conventionally, in vitro testing is performed on human cell lines, then basic in vivo testing is completed in rodents, such as mice and rats. Correlation of results between mice and humans is difficult and unpredictable, at least because mice and humans share approximately 70 percent of the same protein-coding gene sequences, these genes constituting about 1.5 percent of their respective genomes. After testing in rodents, which may or may not be representative of how a human body responds, testing is moved into nonhuman primates. However, numerous nonhuman primates are typically used because of the potential disparity between human cell line testing and in vivo rodent testing. Jumping from in vivo testing in rodents to in vivo testing in humans is not possible in most cases per FDA regulations.

In addition, the U.S. Department of Agriculture reports that 820,812 animals were used in research in the U.S. in 2016, with more than 71,000 of these animals being nonhuman primates. Further, the use of nonhuman primates drastically increased during the 2020 COVID pandemic for vaccine development, resulting in animal shortages worldwide. This shortage was mainly due to the fact that China, as the main source of nonhuman primates to the pharmaceutical industry in the U.S. and elsewhere in the world, stopped exporting NHPs to the U.S. in 2020. Additionally, there is a growing concern in the use of NHPs in biomedical research.

Accordingly, new methods are needed to improve correlation between in vitro and in vivo results, to improve a likelihood of a correlation between preclinical testing and in-human clinical trials, and to reduce the use of nonhuman primates.

Several groups have been trying to use human in vitro iPSCs models to generate and combine in vitro and in silico data for drug discovery. However, methods based on human iPSC in vitro systems lack the in vivo data from humans to verify the accuracy of the in vitro findings. Using personalized iPSCs from NHP matched with each animal, the combinatory approach between in vitro and in vivo in a personalized manner that is currently impossible using human iPSCs could be addressed. Although nonhuman primates share approximately 95% genome homology with humans, it is not a perfect model for human. However, since one can combine both in vitro data from NHP iPSCs and tissues with in vivo data from NHPs (that can be used for in vivo experiments, unlike humans), the combinatory approach is useful for drug discovery.

Further, there are research groups trying to study the process of reprogramming and iPSC generation to understand the process of rejuvenation and aging using human cells and mice. The process of iPSC generation in NHP is challenging and not as straight forward as in humans and mice. Additionally, different species of nonhuman primates have different lifespans. For example, chimpanzees live up to approximately 55 years while macaques live up to approximately 30 years. Comparative study of iPSCs among chimpanzee, human, macaques, and other NHPs will help understand the aging and rejuvenation process.

In general, the methods described herein improve upon conventional methods for preclinical evaluations of drugs and therapeutics in nonhuman primate models before FDA approval. For example, the methods described include using nonhuman primate induced pluripotent stem cells (iPSCs); individualized genome assemblies from the same animal from which the iPSC were derived; and in vivo testing on the same animal from which the iPSCs and individualized genome assemblies were derived for preclinical evaluations to improve results correlation between in vitro and in vivo testing and improve genetic mapping of safety and therapy responses, resulting in more selective drug studies (focusing on those with higher correlation) and reduced nonhuman primate use over time than that of conventional methods for preclinical evaluations of drugs and therapeutics in nonhuman primate models.

In some embodiments, the methods described herein may provide a technical effect and/or advantage of avoiding immune rejection issues because the experiments may be designed to utilize iPSCs, an individualized genome, and autologous tissue and cell transplantation for a specific and individual nonhuman primate (NHP). For example, since the cells and tissues are derived from the specific animals' own cells for various stages of testing, relevant conclusions can be drawn and/or comparisons made that can better inform human testing and anticipated reactions in humans.

As used herein, the terms drug, therapy, pharmaceutical, gene therapy, genome editing tool, treatment, etc. may be used interchangeably in that the methods described herein may be used for preclinical evaluation of any of these and more.

### Practical Applications

The practical applications of the methods described herein include improved methods for preclinical evaluations of: Good Laboratory Practices (GLP); potency, functionality, and drug safety; anti-drug antibody (ADA) responses and related genetics; genome editing methods including potency and functionality; toxicology testing of gene therapies using iPSCs and/or cells or tissues derived from iPSCs; cell therapies including immunotherapy and Chimeric Antigen Receptor (CAR) T-cell for cancer therapies; monoclonal antibodies for cancer and other non-infectious or infectious diseases (providing a faster and nonterminal way to interrogate genetically validated targets); and iPSC labeling for imaging purposes. Various practical applications of the compositions and methods may be further described elsewhere herein.

The methods described herein may be particularly applicable to preclinical evaluation of drugs, therapies, pharmaceuticals, etc. for humans since human DNA is, on average, 96% identical to the DNA of our most distant primate relatives, and nearly 99% identical to our closest relatives, chimpanzees and bonobos.

During drug development, from the preclinical phase through the post-marketing phase, the safety level of a drug is measured. A safety measure usually denotes a type and quantity of adverse events associated with a product's use compared with the expected rate. Good Laboratory Practice (GLP) is designed to promote the development of quality test data. GLP provides tools and methodologies that ensure a sound approach to managing laboratory studies during the preclinical phase of drug development.

To address questions regarding safety measures for GLP, nonhuman primate genomes may be used to establish a possible correlation between a genotype and safety of the drug under testing in the same nonhuman primate. The assembled genome information may resolve whether specific genotypes of nonhuman primates are associated with drug resistance, drug toxicity, and development of antidrug antibodies (ADA) in the same NHP as well as potential issues with a drug and/or a therapy in the same NHP. The assembled genome information can allow creation of genetic panels (e.g., genetic assays to detect one or more genetic mutations or markers) through which one can predict whether a new colony of animals would be fit for a certain drug and/or therapy experiment. An example is the problem of animals developing ADA in the drug preclinical testing. Despite screening animals for existing antibodies against the drug of interest, there are still a population of animals that develop ADA during preclinical testing. Genetic panels based on full genomes of the animals with regards to Immunoglobulin receptor regions, Major Histocompatibility Complex (MHC), Minor Histocompatibility Complex (MiHC), Killer-cell immunoglobulin-like receptor (KIR), and other immune genes would be useful in ruling out the animals that will develop ADA in preclinical testing. Additionally, personalized iPSC lines for each NHP in the colony is an appropriate tool to evaluate the safety of a drug and/or therapy in vitro pre-GLP and/or before using the drug and/or therapy in vivo, resulting in fewer NHP being used.

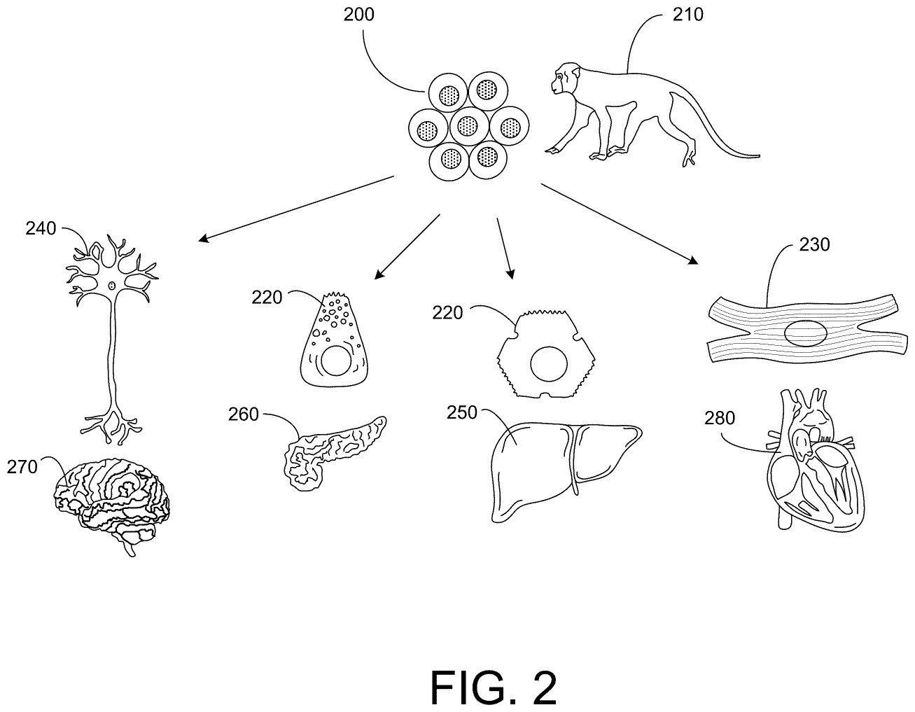

As shown in FIG. 2, iPSCs 200 from a NHP 210 can be differentiated into the main lineages: ectoderm 240, mesoderm 230, and endoderm 220 and then into final tissues, for example liver 250, pancreas 260, neural cells 270, cardiomyocytes 280, hematopoietic lineages, immune cells, etc. using the methods described herein. Thus, the safety of a drug can be initially evaluated through an in vitro assay on tissues derived from individualized animal iPSCs before it is used on the same animal in vivo. The methods described herein may be used to perform pre-analysis steps (e.g., testing) on iPSC-derived cells and tissues that are directly relevant to an individual animal being assessed for safety, the individual animal have high homology to humans. That is, performing the testing and/or pre-analysis of such derived aspects on an individual animal and later using the same animal to experiment upon provides advantages over conventional systems that utilize in vitro testing in a first species to assess safety, etc., in vivo testing in a second species to assess safety, etc., and further yet clinical studies in a target species (e.g., humans) that is different than the first and second species. One example advantage of the methods described herein may include the generation of data that provides a direct correlation between the iPSCs undergoing pre-analysis, the animal undergoing experimentation, and the genetics of the same animal because the exact same individual animal and its respective genome are used for both steps. Put another way, using the same exact individual animal for in vitro and in vivo assessments and genetic relationships provides for a direct correlation of pre-analysis data to experimental results data that can be directly related to humans, given the high homology between humans and NHPs. Example derived aspects may include the potency, functionality, and safety measures associated with the iPSC-derived cells and tissues derived from personalized iPSCs. In particular, such aspects are predictive of potency, functionality, and safety measures to be established for the same animal before in vivo experimentation in the same animal.

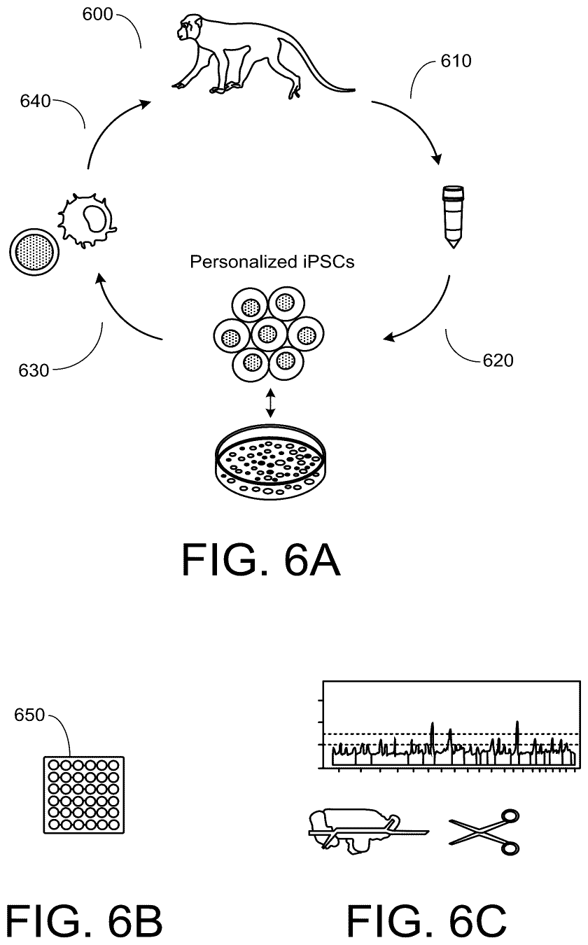

As shown in FIG. 6B, cells and tissues derived from each individual animal's iPSC lines 650 (e.g., cells or tissues) may be labeled through fluorescent proteins or other cell labeling methods and tracked within the animal's body during autologous transplantation. The iPSC-derived cells and tissues, as shown in FIG. 2, can be labeled and are viable tools to conduct autologous transplant or injection into the same animal to evaluate functionality of the cells, as shown in FIG. 6A. In some embodiments, a preclinical testing method using the methods and compositions described herein includes autologous transplantation, into a first NHP, of labeled cells and tissues derived from iPSCs from the first NHP. The preclinical testing method may further include imaging of the cells. Imaging may be used to determine trafficking patterns, localization, efficacy, cell or tissue viability, and other parameters. Labeling and imaging iPSC-derived cells and tissues, that are transplanted into the same animal, can be used to analyze effects of treatments that are being tested on the NHP. For example, labeling and imaging allows the transplanted cells to later be isolated after a predefined time period, for example using fluorescence activated cell sorting, to track the cells in vivo, to assess cell viability, among other assessments. Using the same nonhuman primate during various steps of preclinical treatment testing can ensure a 1:1 assessment and/or correlation for safety, efficacy, etc. In addition, using the same nonhuman primate for various steps of preclinical treatment testing can result in fewer NHP being used.

### Methods and Compositions

### Overview

In general, described herein are methods for deriving induced pluripotent stem cells (iPSCs) from nonhuman primates. For example, nonhuman primates include cynomolgus macaques, pig-tailed macaques (e.g., Macaca nemestrina, Macaca leonine, Macaca sylvanusi), baboons, African green monkeys, rhesus macaques, chimpanzees, and the like.

In general, described herein are methods for sequencing individualized nonhuman primate genomic DNA and de novo assembly of the genome fragments of the nonhuman primates that have been sequenced, resulting in an individual reference genome of an individual nonhuman primate. For example, the assembly of genome fragments may include using long-read technologies, but may also include short-read technologies, as described elsewhere herein.

As used herein, medium may include, but is not limited to, DMEM medium, RPMI™ medium, F12™ medium, and the like. In some embodiments, specialty media are detailed below or elsewhere herein.

An embodiment of a method for preclinical evaluation includes deriving induced pluripotent stem cells (iPSCs) from at least one individual of a population comprising a single species of nonhuman primate; and generating an individual reference genome for the at least one individuals. In such embodiments, the same at least one individual (i.e., nonhuman primate) is then used in subsequent preclinical testing and the in vivo results from the same nonhuman primate are compared to the in vitro results for the iPSCs from same nonhuman primate. The solution provided by the method is that using iPSCs and a reference genome from the same nonhuman primate provide a strong foundation for pre-clinical testing that reduces the risk level of in vivo testing.

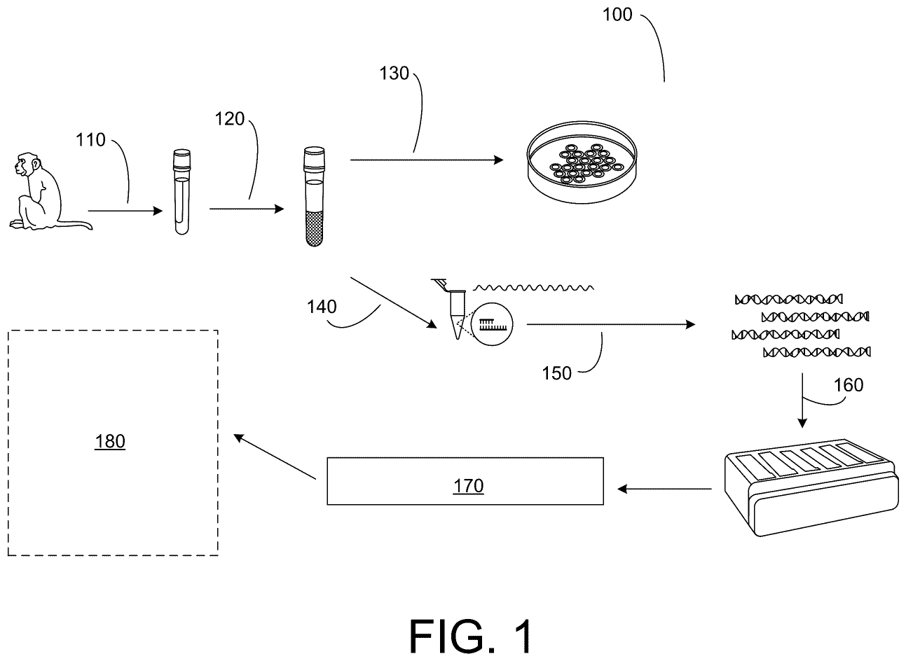

FIG. 1 is a schematic showing an embodiment of a method for preclinical testing of human therapies using nonhuman primate (NHP) iPSCs, genome assembly, and related genomic analyses and/or computations. For example, a method 100 may include: obtaining tissue or blood from a first nonhuman primate 110; isolating PBMCs from the first nonhuman primate 120; deriving iPSCs from the PBMCs from the first nonhuman primate 130; extracting DNA from the PBMCs from the first nonhuman primate 140; preparing a DNA library 150 using, at least in part, a DNA sequencing method 160; preparing a full genome assembly for the first nonhuman primate 170; and optionally (shown by dashed lines), performing computational analysis 180. An example of the genomic analyses is in the area of gene therapy preclinical testing. In the process of gene therapy, an individualized genome is desirable to design guide RNAs (gRNA) to avoid off-target effects and to accurately insert target genes in the target locus using AAV vectors by accurate design of homology arms in AAV vectors. A personal genome of the NHP matched with the iPSCs from the same animal is the ideal tool for preclinical testing of the gene therapies and other cell therapies like CAR-T cells that require individualized genome information such as T Cell Receptor information. Computational analysis may include identification of Major and/or Minor Histocompatibility complex genes, and/or immune system-related genes. Computational analysis may include anti-drug antibody (ADA) response genotyping. An analysis may include performing preclinical genotyping. In an example embodiment, the method may further include performing in vitro testing on the derived iPSCs from the first nonhuman primate; and correlating results from the in vitro testing with a genotype of the first nonhuman primate. An example is liver toxicity in animals and humans due to AAV vectors or other cell gene and therapy methods. Initial testing on iPSC derived liver cells or immune cells (e.g., PBMCs) to predict the level of immunogenicity of the AAV vector in vitro may be used as a predictor of liver toxicity in NHP in vivo. In vivo liver toxicity may be predicted based on correlating the in vitro results with a genotype of the same NHP. In an embodiment, the method may include performing in vivo testing in the first nonhuman primate; and correlating results from the in vivo testing with results from the in vitro testing and/or with the genotype of the first nonhuman primate.

Nonhuman Primate iPSC Generation

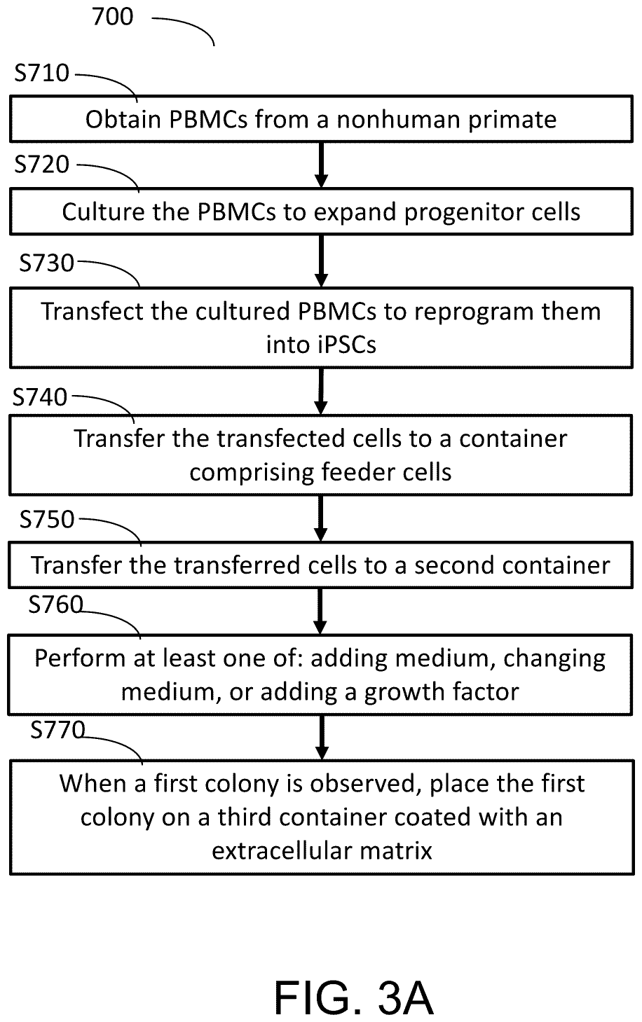

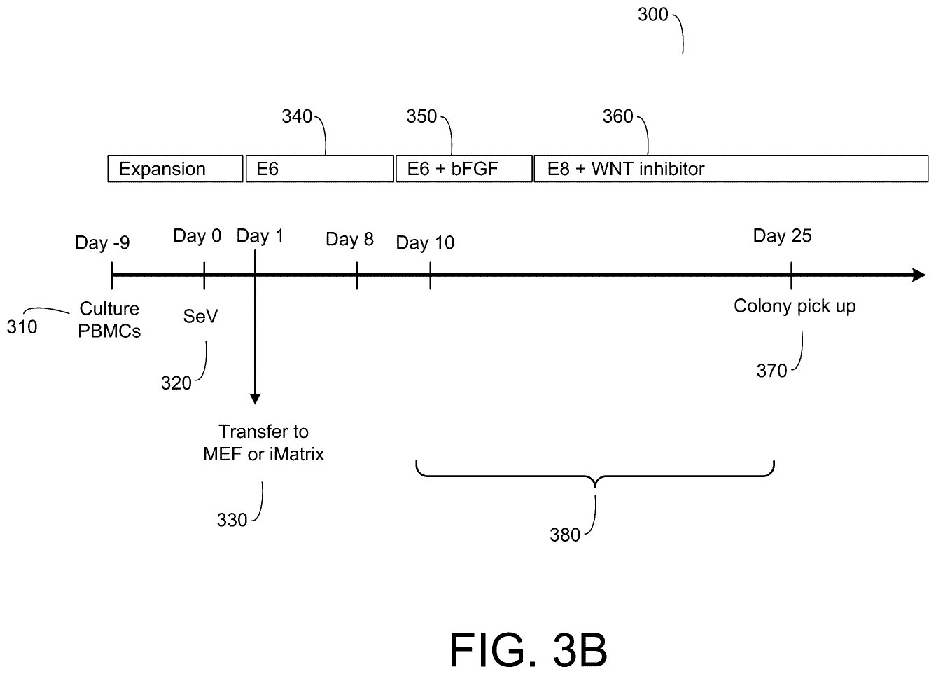

FIG. 3A shows an embodiment of deriving iPSCs from nonhuman primate PBMCs. The method 700 includes: obtaining peripheral blood mononuclear cells (PBMCs) from at least one individual of a population comprising a single species of nonhuman primate S710; culturing the PBMCs to expand blood progenitor cells (e.g., including CD34+ cells) in a hematopoietic stem cell (HSC) expansion medium for a pre-determined time period S720; transfecting the cultured PBMCs to reprogram the cultured cells into iPSCs S730; transferring the transfected cells into a container comprising a plurality of feeder cells S740; transferring the cells to a second container S750; on selected days after transfer to the second container, performing at least one of: adding medium to the cells; changing the medium in the cells; or adding one or more cell growth factors to the cells S760; and when a first cell colony appears; passaging the cells by placing each colony in a third container coated with an extracellular matrix S770.

The method 700 may further include expanding the first colony for use in one or more of: an in vitro test, an in vivo test, or creation of a genome library for the individual primate.

In some embodiments, transfecting includes transfecting with a combination of transcription factors so that the cells are induced to overexpress the transcription factors. For example, the transcription factors may include: c-myc, KLf4, Sox2, or Oct3/4. Additional or alternative transcription factors may be used and are within the scope of the present disclosure. For example, transcription factors such as Klf2, Nanog, Tfcp2L1 and Stat3 may additionally or alternatively be used.

In some embodiments, the expanding may include washing the iPSCs with a buffer (e.g., phosphate-buffered saline) and incubating the iPSCs with a cell detachment solution, for example trypsin, a chelating agent, collagenase, or the like. In some embodiments, the incubating occurs at about 37 degrees Celcius for a predetermined time period. The predetermined time period may be about 30 seconds to about 10 minutes; about 1 minute to about 60 minutes; about 1 minute to about 5 minutes; etc.

In some embodiment of method 700, transferring the transfected cells to a container with feeder cells occurs no more than about one day post-transfection. The feeder cells may comprise mouse embryonic fibroblast (MEF) cells, SNL feeder cells, or the like.

In some embodiments, transferring the cells to the second container occurs no more than about one day after transferring to the container with the feeder cells.

In some embodiments, the expanding comprises aspirating and dissociating the iPSCs into a single cell suspension.

In an example application of method 700, as shown in FIG. 2, the iPSCs may be differentiated into main lineages. The main lineages may include at least one of: ectoderm 240, mesoderm 230, and endoderm 220. Additionally, or optionally, the main lineages may be further differentiated into final tissues. The final tissues may be used for in vitro testing, autologously translated into the first nonhuman primate for in vivo testing, and/or used in genome library generation.

The method 700 may further include generating an individualized genome assembly of the at least one individual, the same individual from which the PBMCs were harvested or obtained. Generating the genome assembly may include extracting a quantity of high-molecular weight (HMW) DNA from the obtained PBMCs by means of a process for purification of archival quality DNA, as described elsewhere herein; sequencing long-read fragments of the HMW DNA; and assembling and annotating the long-read fragments to recreate the genome of the at least one individual from which the PBMCs were obtained.

In an embodiment, a method for pre-clinical drug evaluation may include: an individualized genome assembly for a test subject; and induced pluripotent stem cells (iPSCs) derived from the test subject. Cells and/or tissues derived from iPSCs the test subject may be implanted iPSCs into the test subject to evaluate functionality of the test subject's cells

In an embodiment, one or more panels may be generated for predicting whether a particular animal or community of animals is a fit for a certain drug experiment based on the one or more panels. For example, these panels include testing small molecules or gene therapy AAV vectors on iPSC-derived tissues (e.g., liver cells) and/or primary cells (e.g., PBMCs).

In an embodiment, the cells and/or tissues derived from the iPSCs from the test subject may be used to generate guide RNA or a gene-editing tool. The guide RNA or gene editing tool may be used to perform in vivo testing of the guide RNA or gene-editing tool in the test subject.

FIG. 3B shows an embodiment of a method 300 for deriving iPSCs from nonhuman primate PBMCs. For example, peripheral blood mononuclear cells (PBMCs) are obtained from an individual from a species of nonhuman primate, and the PBMCs are isolated from the animals' whole blood. The PBMCs may be isolated from the animals' whole blood using density gradient centrifugation (DGC), fluorescence active cells sorting, magnetic bead-based separation, buoyancy activated cell sorting (BACS), and the like. In an embodiment, PBMCs are isolated using DGC. In an embodiment, PBMCs are isolated using BACS.

In some embodiments, a method 300 for reprogramming NHP PBMCs into iPSCs includes culturing 310 the PBMCs to expand blood progenitor cells (e.g., CD34+ cells) in a hematopoietic stem cell (HSC) expansion medium such as StemSpan™ (Stemcell Technologies Inc., Vancouver, British Columbia, Canada) for a period of about three days to about 10 days, about 5 days to about 10 days, about 6 days to about 10 days, about 7 days to about 10 days, about eight days to about 10 days, or about nine days. In some embodiments, an alternative for StemSpan™ may be used. For example, a composition for the expansion of hemopoietic stems (HSPC) and/or progenitor cells of nonhuman primates may include IL3, IL6, FLT-3, TPO, and SCF (e.g., prior to transfection with reprogramming transcription factors). In some embodiments, animal serum (e.g., Fetal Bovine Serum) in the expansion medium may be replaced or combined with poly vinyl alcohol (PVA) to improve the efficiency of expansion of HSPC from nonhuman primates. CD34 is expressed not only by HSCs but by a multitude of other non-hematopoietic cell types, including muscle satellite cells, corneal keratocytes, interstitial cells, epithelial progenitors, and vascular endothelial progenitors. Thus, several different cell types may potentially exhibit progenitor activity and be expanded

Further, a method 300 for reprogramming PBMCs into iPSCs may include transfecting blood progenitor cells with transcription factors 320. The NHP PBMCs are reprogrammed into NHP induced pluripotent stem cells (iPSCs). The method for reprogramming the PBMCs into iPSCs may include transfecting the PBMCs with one or more transcription factors so that the cells are induced to overexpress the transcription factors. Transcription factors are one of the groups of proteins that read and interpret DNA. They bind to the DNA and help initiate a program of increased or decreased gene transcription. The stem cell derivation protocol is optimized for nonhuman primates specifically as there are different approaches taken to generate optimized stem cells for nonhuman primates. In some embodiments, transcription binding sites of these transcription factors may be mapped on individualized primate genomes to improve efficiency of the reprogramming process in NHPs, especially since transcription binding sites in human genomes are not completely conserved in NHP species. Thus, the use of human iPSCs reprogramming kits is not efficient for iPSC generation in NHP species. This is also important considering the fact that NHP genomes are very heterogenous and existing mutations in NHP genomes result in inefficient binding of transcription factors (from human iPSC generation kits) in nonhuman primate cells.

Various transfection methods may be used, including but not limited to: electroporation, calcium-phosphate exposure, liposome-based transfection, viral-mediated transfection (also known as transduction), and the like. In an embodiment, a method for transfecting PBMCs includes transduction. Transduction may be accomplished using, for example, a Sendai virus, an adenoviral, an oncoretroviral, or a lentiviral vector. In some variations, transfection includes using a Sendai virus vector. In some instances, transfecting PBMCs includes liposome mediated transfection. For example, the transcription factors may include: c-myc, KLf4, Sox2, or Oct3/4. Additional or alternative transcription factors may be used and are within the scope of the present disclosure.

Further, a method for reprogramming PBMCs into iPSCs may include co-culturing the cells (e.g., transfected and untransfected) with feeder cells 330. The feeder cells may comprise mouse embryonic fibroblast (MEF) cells, SNL feeder cells, or the like. The ratio of transfected cells to feeder cells may be about 1:2 to about 1:8, about 1:3 to about 1:7, about 1:4 to about 1:6, for example about 1:5. Co-culturing may occur on a subsequent day after transfection, for example about 20 hours to about 36 hours, about 18 hours to about 30 hours, about 18 hours to about 36 hours, about 22 hours to about 26 hours, etc. after transfection.

Co-culturing may occur in Essential-8™ medium (Thermofisher Scientific). In an embodiment, the Essential-8™ medium may include a tankyrase ½ inhibitor. The tankyrase ½ inhibitor may be present in the medium at a concentration of about 1 μM to about 3 μM, about 1.5 μM to about 2.5 μM, about 1 μM to about 2.5 μM, about 1.5 μM to about 3 μM, about 1.75 μM to about 2.25 μM, etc. In some embodiments, the tankyrase ½ inhibitor may maintain the NHP iPSCs in a pluripotent state and significantly reduce spontaneous differentiation of the NHP iPSCs.

Further, a method 300 for reprogramming PBMCs into iPSCs may include transferring unadhered cells to new medium. For example, the transfer may occur about 24 hours to about 72 hours, about 18 hours to about 78 hours, about 18 hours to about 30 hours, about 30 hours to about 42 hours, about 42 hours to about 52 hours, about 52 hours to about 66 hours, about 66 hours to about 78 hours, etc. after co-culturing. The unadhered cells may be cultured in a basal medium, for example MEM medium, DMEM medium, RPMI medium, F12 TM medium, and the like. Further, a method 300 for reprogramming PBMCs into iPSCs may optionally include adding medium to the adherent cells 340. The added media may comprise a medium that is feeder-free and xeno-free that supports the reprogramming of somatic cells and the spontaneous or directed differentiation of pluripotent stem cells (PSCs). For example, the added medium may be Essential-6® medium (Thermofisher Scientific). In an embodiment, adding medium occurs about 48 hours to 96 hours, about 42 hours to about 102 hours, etc. after transfection.

Further, a method 300 for reprogramming PBMCs into iPSCs may include optionally replacing the medium on the adherent cells. The medium may be replaced after about 90 hours to about 144 hours, about 96 hours to about 144 hours, about 90 hours to about 126 hours, about 114 hours to about 126 hours, etc. after transfection. The replacing medium may be a medium that is feeder-free and xeno-free that supports the reprogramming of somatic cells and the spontaneous or directed differentiation of pluripotent stem cells (PSCs). For example, the added medium may be Essential-6® medium (Thermofisher Scientific).

Further, a method 300 for reprogramming PBMCs into iPSCs may include optionally replacing the medium with medium comprising one or more growth factors 350. For example, the medium may comprise a medium that is feeder-free and xeno-free that supports the reprogramming of somatic cells and the spontaneous or directed differentiation of pluripotent stem cells (PSCs). For example, the added medium may be Essential-6® medium (Thermofisher Scientific). The growth factor may comprise basic fibroblast growth factor, DJ-1, epidermal growth factor, and the like. In an embodiment, the growth factor comprises basic fibroblast growth factor. The concentration of the growth factor (i.e., bFGF) may be about 100 ng/ul to about 200 ng/ul. In some embodiments, this particular growth factor and/or concentration is advantageous for the NHP iPSC derivation process since commercially available human bFGF is not as efficient in cross-reactivity with NHP cells. Medium replacement may occur about 144 hours to about 192 hours, about 160 hours to about 250 hours, about 162 hours to about 174 hours, about 188 hours to about 198 hours, about 210 hours to about 222 hours, about 232 hours to about 246 hours, etc. after transfection. Medium replacement may be repeated daily for one or more days or a plurality of days, for example repeated once, twice, thrice, or more.

Further, a method 300 for reprogramming PBMCs into iPSCs may include optionally replacing the medium daily for one or more days or for a plurality of days 380. For example, the medium may be replaced between about 240 hours post-transfection to about 440 hours post-transfection. The medium may be replaced with basal medium, Essential-6®, Essential-8®, a medium comprising tumor growth factor-b (TGF-b) and/or b-FGF, or similar medium. In some embodiments, the medium may be replaced daily until visual identification of a first colony of iPSC.

Further, a method 300 for reprogramming PBMCs into iPSCs may include culturing the first colony of iPSC on a matrix 370, for example an extracellular matrix, Matrigel®, Cultrex®, Geltrex®, and the like. The first colony may be cultured in medium comprising a serine/threonine kinase inhibitor. For example, the medium may include Essential-8®. The serine/threonine kinase may include a Rho inhibitor or a Rho-associated coiled-coil containing protein kinase (ROCK) inhibitor, for example, Thiazovivin (TZV) or similar products. A concentration of the inhibitor may be about 1 μM to about 3 μM.

Further, a method 300 for reprogramming PBMCs into iPSCs may include passaging the cells after about 12 hours to about 48 hours. Passaging 360 may include refreshing the medium and adding a Wnt pathway inhibitor, tankyrase½ inhibitor, or the like. The inhibitor may be at a concentration of about 1 μM to about 3 μM, about 1.5 μM to about 2.5 μM, about 1 μM to about 2.5 μM, about 1.5 μM to about 3 μM, about 1.75 μM to about 2.25 μM, etc.

Optionally, iPSCs may be passaged and/or expanded by washing with a buffer (e.g., phosphate buffered saline) and incubating the cells with a cell detachment solution. The cell detachment solution may comprise collagenase (or recombinant enzymes thereof), trypsin (or recombinant enzymes thereof), a chelating agent (e.g., ethylenediamine tetra-acetic acid), and the like.

Optionally, iPSCs may be expanded or maintained by culturing in medium on feeder cells or an extracellular matrix, as described elsewhere herein. The medium may comprise a serine/threonine kinase may comprise a Rho inhibitor or a Rho-associated coiled-parenthesis coil containing protein kinase (ROCK) inhibitor. The inhibitor may be at a concentration of about 1 nM to about 3 nM, about 1.5 μM to about 2.5 μM, about 1 μM to about 2.5 μM, about 1.5 μM to about 3 μM, about 1.75 μM to about 2.25 μM, etc.

In embodiments with subsequent passaging, the iPSCs may be incubated in a container, without feeder calls, the container having a substrate coated thereon. The substrate may be configured to retain pluripotency of the cells, a stable karyotype of the cells, and/or an expression of pluripotency markers by the cells. For example, the substrate may comprise laminin, a recombinant laminin fragment (e.g., 511 e8 fragment), vitronectin, recombinant vitronectin, or a combination thereof. The medium may comprise Essential-8®, E8-specific supplement (e.g., TGF-b, bFGF, etc.), and a Wnt pathway inhibitor, tankyrase1/2 inhibitor, or the like. The inhibitor may be at a concentration of about 1 nM to about 3 nM, about 1.5 μM to about 2.5 μM, about 1 μM to about 2.5 μM, about 1.5 μM to about 3 μM, about 1.75 μM to about 2.25 μM, etc. The subsequent passaging may result in colonies with little-to-no differentiation.

### Genome Assembly Using Primate High Molecular Weight (HMW) DNA and Long-Read Sequencing

Returning to FIG. 1, which includes extracting DNA from the PBMCs from a first nonhuman primate 140, preparing a DNA library 150 using, at least in part, a DNA sequencing method 160; and preparing a full genome assembly for the first nonhuman primate 170. Although PBMCs are shown and described in FIG. 1, other cells or tissues, such as, for example neuronal progenitor cells, keratinocytes, hepatocytes, B cells, or fibroblasts may be used. Genomic DNA having large numbers of base pairs is known as high-molecular weight DNA because the considerable number of base pairs in a long-read results in a DNA fragment having a high molecular weight in contrast with a short read having many fewer base pairs and thus being of a lower molecular weight. Typically, a long read may include at least about 10,000 base pairs but may also be between about 5,000 base pairs to about 50,000 base pairs. Although long reads are described, short reads are also contemplated herein.

To conduct a personalized genome assembly on a nonhuman primate animal colony, which matches with the individualized iPSCs lines (described above), the DNA is extracted using a process for purification of archival quality DNA, as shown at block 140. DNA maybe extracted using enzyme-based extraction or chloroform based extraction to avoid mechanical breaking of the cells and/or DNA. In an embodiment, a protocol embodied in the Gentra® Puregene® Cell Kit (QIAGEN, INC., Germantown, MD, USA) may be employed to extract the DNA, for example high molecular weight DNA. One of ordinary skill will recognize that other protocols for extracting the DNA may be applicable for example, the protocol embodied in the QuiAmp® genomic DNA kits (QIAGEN, INC., Germantown, MD, USA).

As shown in FIG. 1, the method 100 includes preparing the DNA library using, at least in part, a DNA sequencing method 160. DNA sequencing may include using long-read sequencing technologies, such as, for example, those that directly sequence single molecules of DNA in real-time, optionally with or without amplification (e.g., those available from Illumina®, PacBio®, Nanopore®). Other methods of long-read sequencing may also be used for the purpose of genome assembly leveraging long-read methods.

In some embodiments, library preparation and sequencing may resolve more variable regions of the genome such as in the region of the Major Histocompatibility Complex (MHC) locus. Further, in an embodiment, genomic DNA above a predefined base pair threshold may be purified based on size, for example using beads, electrophoresis and purification, and the like. The purified DNA may be used for genome assembly pipelines, at block 170 of FIG. 1, using software-based assemblers for single molecule sequencing tools. For example, any of a variety of assembly tools, for example Flye, Redbean, Canu, and the like, may be used to draft a genome assembly from the purified DNA sequences.

In some embodiments, an assembled genome may be used in conjunction with the corresponding iPSCs line (from the corresponding animal) to design guide RNA (that fits individual animals based on the corresponding single nucleotide polymorphisms (SNPs) for that animal). The guide RNA may be used in combination with gene editing method such as Clustered Regularly Interspaced Short Palindromic Repeats (CRISPR) or Adeno-Associated Virus (AAV)-based gene editing methods.

### Use of Full Genome Long-Read Sequencing to Predict Formation (or Magnitude) of ADA (Anti-Drug Antibody) Response

In some embodiments, a whole genome-based study may be used for identification of the NHPs that develop anti-drug antibodies (ADA). Some animals develop anti-drug antibodies during the process of evaluating a drug's safety. ADA binds to a drug and thereby masks the potential effect of a drug. Detecting antibodies against a drug using ELISA or other similar methods for detection of antibodies may be performed in a nonhuman primate after the injection of a drug into the NHP. Some animals are genetically more susceptible to develop ADA. Such genetic regions may be identified by conducting whole genome sequencing of the animals that develop ADA compared to those animals that do not develop ADA. Given the genetic variability of nonhuman primates, these identified genetic regions that may contribute to ADA responses in NHP may be correlated to similar genetic regions in humans.

Whole genome sequencing assembly of individual animals may be used to predict possible Anti-Drug Antibody (ADA) response in animals. In an embodiment, phenotypic data collected from an animal that develops ADA may be correlated with the corresponding whole genome data for the respective animal to screen for candidate genetic loci that correlate with ADA. Specifically, reconstruction of the variable immune regions, including but not limited to, B Cell receptor (BCR) genomic loci and immunoglobulin coding regions, Major Histocompatibility (MHC) region, and possibly genomic loci for T Cell receptors regions may identify the underlying genetic components of ADA in some animals that can be resolved via whole genome information data. One limitation of such approach has been the use of short-read sequencing which makes it challenging to reconstruct or assemble these long variable regions in the genomes of NHP. A long-read sequencing approach can be used to assemble these variable regions of the innate and adaptive immune system (including BCRs, MHC, KIR) to find correlation of ADA and animals' genotypes. Subsequently, these personal genomes can be used for profiling each animal's B Cell Receptor and T Cell Receptor repertoire using single-cell RNA sequencing and aligning these regions to the personal genome to help predict ADA for each animal.

In some embodiments, one or more genetic assays may be developed based on genetic variations in genomic regions of B Cell Receptors (BCR) and other candidate regions (loci) using genome wide association studies (GWAS) with a focus on target loci of interest.

If there are retrospective data of ADA response(s) in the colony animals, such information may be used to correlate with the genotype identified by the colony whole genome assemblies.

Alternatively, if there is no record of ADA response from the animal colony, genome information may be used to track animals over time for future studies and investigate whether the animal will develop ADA. In such embodiments, the phenotypic information may be correlated to the assembled genotypes of each animal.

Full Genome Assembly and Annotation of the Individual Animals within the Colony for Subsequent Application of Individualized Medicine and Drug Safety Testing in Nonhuman Primates

In some embodiments, the PBMCs isolated at block 130 in FIG. 1 may be used to assemble individual genomes for each NHP within the colony, at block 170, which may be matched to the corresponding NHP from which iPSCs were derived. The technical effect and/or advantage of having a personalized genome for an NHP is that autologous tissue and cell transplantation can be conducted on the animals without immune rejection. The cells and tissues are derived from the specific animals' own cells so the Major Histocompatibility Complex (MHC) and any other immune components pertaining to transplant immune rejections match the cells being injected. Additionally, the individualized genomes from each animal may enable the design of specific guide RNA for future gene therapy or gene editing or cell-labeling purposes with minimal off-target effects. Since iPSCs have self-renewal for many passages, they can be used for many cell culture passages to optimize the guide RNA and evaluate potency and functionality of gene therapy vectors (e.g., AAV vectors), leveraging the individualized genomes.

### Evaluating Drug Safety in Complex Nonhuman Primate In Vitro Models.

To evaluate the measures of a drug and/or therapy safety for GLP, the genomes of the animals are useful for correlation between the genotype and safety of the drug and/or therapy. The generated genome information can resolve such questions and can create panels through which one can predict whether a new colony of animals would be fit for a certain drug and/or therapy experiment.

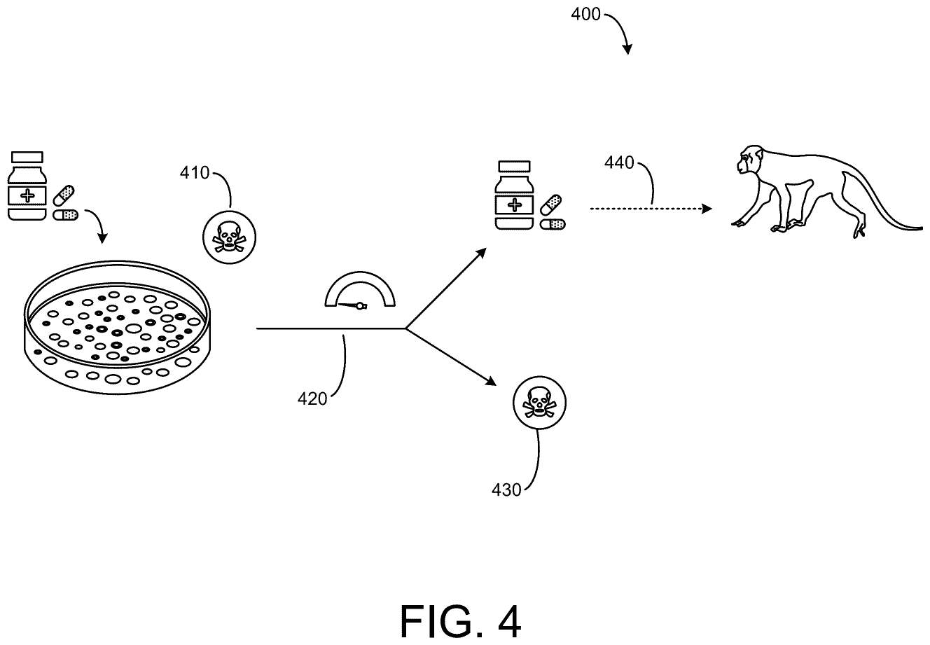

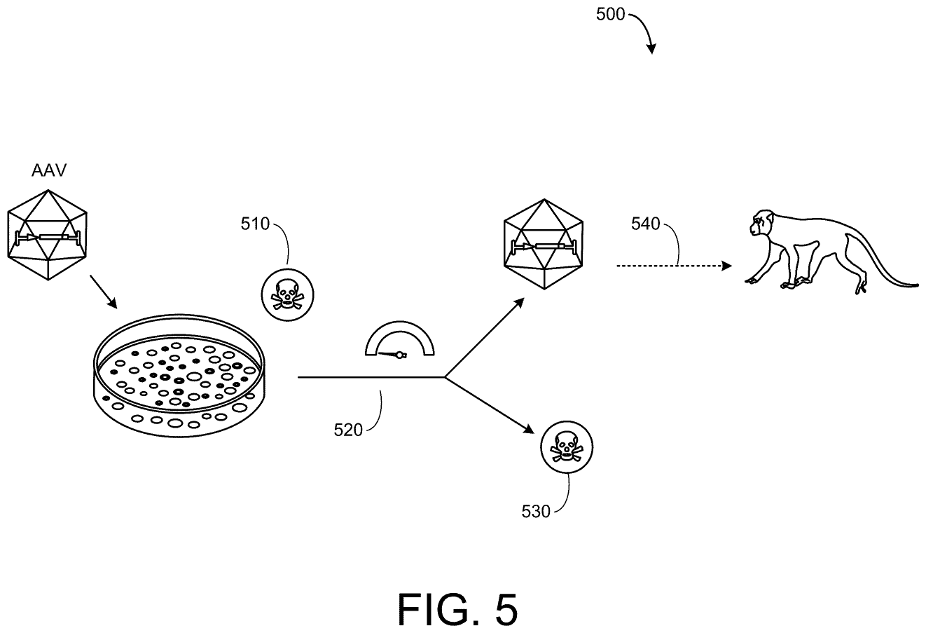

Additionally, a personalized iPSC line for each animal is an appropriate tool to evaluate the safety of the drug in vitro pre-GLP and/or before using the drug in vivo as shown in FIGS. 4-5.

As shown in FIG. 2, iPSCs can be differentiated into a plurality of lineages (ectoderm 240, mesoderm 230, and endoderm 220) and final tissues (e.g., liver 250, neural cells 270, hematopoietic lineages and immune cells, pancreatic cells 260, etc.) so the safety of the drugs and/or therapy can be evaluated on such tissues derived from individualized animal iPSCs and through an in vitro assay on tissues derived from iPSCs before it is used on animals in vivo. The safety measures on tissues derived from personalized iPSCs is predictive of safety in animals before in vivo experiments, as shown in FIGS. 4-5.

For example, as shown in FIG. 4, a method 400 for evaluating safety of a drug is shown. The method for evaluation of potency, functionality, safety, and/or toxicity of a therapy in vitro uses individual iPSCs coupled with individual reference genomes before using that therapy in live animals. The method 400 includes: performing an in vitro toxicity assay on iPSCs derived from a first NHP 410; analyzing results from the in vitro toxicity assay 420; when the results suggest toxicity (e.g., results meet or exceed a predefined threshold), discontinuing the in vitro toxicity assay or changing one or more parameters of the in vitro toxicity assay 430; and when the results suggest limited toxicity (e.g., below a predefined threshold), performing an in vivo toxicity assay in the first NHP from which the iPSCs were derived 440. In vitro toxicity assays may include, but are not limited to: cytotoxicity assays (e.g., ATP based), cell viability assays (e.g., using 3-(4,5-dimethylthiazol-2-yl)-2,5-diphenyltetrazolium bromide (or MTT); 3-(4,5-dimethylthiazol-2-yl)-5-(3-carboxymethoxyphenyl)-2-(4-sulfophenyl)-2Htetrazolium (or MTS), neutral red, etc.); inflammatory response assays (e.g., using enzyme-linked immunosorbent assays (ELISA)); cell interaction assays (e.g., using multi-compartment perfused systems, etc.); and the like. In vivo toxicity tests may include but are not limited to: assessing lethality of a drug (e.g., acute lethality, subacute, subchronic, chronic, etc.); running proteomic, metabolomic, and/or toxicogenomic studies and assays; and the like.

Further for example, as shown in FIG. 5, a method 500 for assessing a potency of a gene editing tool includes: performing an in vitro potency assay on iPSCs derived from a first NHP 510; analyzing results from the in vitro potency assay 520; when the results suggest potency below a predefined threshold, discontinuing the in vitro potency assay or changing one or more parameters of the in vitro potency assay 530; and when the results suggest potency meeting or exceeding the predefined threshold, performing an in vivo potency assay in the first NHP from which the iPSCs were derived 540.

In another embodiment, a method 500 for assessing a toxicity of a gene editing tool includes: performing an in vitro toxicity assay on iPSCs derived from a first NHP 510; analyzing results from the in vitro toxicity assay 520; when the results suggest toxicity (e.g., toxicity meeting or exceeding a predefined threshold), discontinuing the in vitro toxicity assay or changing one or more parameters of the in vitro toxicity assay 530; and when the results suggest toxicity (e.g., toxicity below the predefined threshold), performing an in vivo toxicity assay in the first NHP from which the iPSCs were derived 540.

In another embodiment, the gene editing tool includes an adeno-associated vector (AAV). In another embodiment, the gene editing tool includes a CRISPR-based tool.

Individualized genomes for animal colonies provide accurate genomic information for CRISPR-based gene editing including, but not limited to, guide RNA and/or AAV designs. In contrast to the human genome, the publicly available reference genomes for most NHP species, such as cynomolgus macaques, are poorly annotated. Additionally, since within-species genetic variations are high in most NHP (e.g., cynomolgus macaques), a single reference genome would not provide the most accurate information for designing guide RNA for genome editing purposes. The heterogenous individuals within one NHP species such as cynomolgus macaque may result in off-target effects of genome editing. Personalized genome and iPSCs for the animal colony may provide complementary workable tools for accurate design of the guide RNA and avoidance of an off-target effect. The guide RNA design may be conducted either on iPSCs, iPSCs-derived cells, or primary cells such as PBMCs in vitro (using a personalized genome of the animals). This approach may result in accurate/personalized designs of genome editing tools such as guide RNA and AAV homology arms that foster the GLP in in vivo studies and improved potency and functionality of AAV, while reducing the cost of testing and minimizing the use of live animal testing, and/or saving animal lives and the cost of therapy development.

In an embodiment, a method of evaluating the safety and/or toxicity of a drug or gene therapy tool includes: measuring a level of an immune response in peripheral blood mononuclear cells (PBMCs) in vitro (e.g., an immune response to an AAV vector that is used for gene therapy). The immune response generated by PBMCs (e.g., in response to AAV vector) may be measured through quantitative Polymerase Chain Reaction (qPCR), bead-based assays (e.g., Luminex®), and/or ELISA. The method may further include: measuring a level of an immune response in PBMCs in vivo. The expression of some innate immune genes such as Toll-Like Receptors (TLRs) and Interleukins may predict the level of toxicity of the AAV vector in a living animal.

Preclinical Testing of Drugs or Therapies on the Same Animal (Autologous Therapies) Using iPSCs and Personalized Genome

In an embodiment, cells and tissues derived from each individual animal's iPSCs lines may be tracked through in vivo imaging methods. For example, as shown in FIG. 6B, iPSC-derived cells and tissues 650 may be labeled (e.g., with fluorescent proteins or other markers). As shown in FIG. 6A, the labeled iPSC-derived cells or tissues may be autologously transplanted or injected into the same animal to evaluate functionalities of the cells.

Individualized iPSCs for each animal in a colony provide a tool to label/edit the iPSCs for demonstration of safety or level of therapeutic efficacy or optimizing gene editing for any purpose. The data from the in vitro assays may be used as a preliminary assay before in vivo testing in the animal. Such assays include the evaluation of immune responses of PBMCs or other cells/tissues to a stimulus (e.g., AAV), as described elsewhere herein. Successful gene edited iPSCs lines (with safe toxicity evaluation results on the target cells/tissues) may be used for further in vivo testing and/or later autologous transplantation of the cells in the animal for imaging and functional assays, as shown in FIGS. 4-6C.

For example, a method 600 of autologous transplantation of NHP iPSC-derived cells or tissues includes: obtaining tissue or blood from at least one individual of a population comprising a single species of nonhuman primate 610; deriving iPSC from the tissue or blood 620; differentiating the iPSC into a tissue or cell type of interest 630; and autologously transplanting the tissue or cell type of interest into the at least one individual 640.

In some embodiments, method 600 further includes: performing in vitro testing on a subset of the derived iPSCs from the at least one individual. In vitro testing may include cell viability assessments, treatment efficacy assessments (e.g., target protein inhibition or activation, target pathway inhibitor or activation, cell cycle inhibition or activation, etc.), cell toxicity assessments, etc.

In some embodiments, autologously injecting or transplanting includes autologously injecting or transplanting a second subset of the derived iPSCs from the at least one individual into the at least one individual includes performing in vivo testing in the at least one individual; deriving in vivo testing results from the in vivo testing; and correlating the in vivo testing results with the in vitro testing results. An example is liver toxicity in animals and humans due to AAV vectors or other cell and gene therapy methods. Initial testing on iPSC derived liver cells or immune cells (e.g., PBMCs) to predict the level of immunogenicity of the AAV vector in vitro may be used as a predictor of liver toxicity in NHP in vivo.

A first subset of derived iPSCs may be during a first time period of iPSC expansion in vitro while a second subset of derived iPSCs may be during a second time period of iPSC expansion in vitro.

In an embodiment, method 600 may include performing gene editing on the derived iPSC or the differentiated tissue or cell type of interest, as shown in FIG. 6C and described elsewhere herein.

Individualized iPSCs provide a faster and nonterminal way to interrogate genetically validated targets in NHP. NHP iPSCs have two key features: self-renewal and pluripotency.

For self-renewal, iPSCs can be passaged and expanded in vitro many times over again.

For pluripotency, iPSCs can be differentiated into different tissues and cells. For example, iPSCs may be used to design guide RNA (gRNA) and perform gene editing studies using different tools including AAV, as shown in FIG. 5, which may pertain specifically to NHP species (e.g., cynomolgus macaques and rhesus macaques) that have highly heterogenous within-species populations. Individualized iPSC lines coupled with animal individualized assembled genomes supply the viable tools for a faster nonterminal way to interrogate genetically validated targets in the NHP. Such assays are useful before conducting in vivo experiments.

Because of their self-renewal ability (immortality for many in vitro passages) and their pluripotency features, iPSCs are a workable tool for designing guide RNA and gene editing studies using different tools including AAV (adeno-associated virus). This may specifically apply, for example, to NHP species (cynomolgus macaques and rhesus macaques) that have highly heterogenous populations.

Finally, a robust iPSCs and genomics tool that includes a) individualized iPSCs lines and b) individualized genome assemblies may provide an in vitro system to measure drug safety levels, for design of the gene-editing tools that might otherwise cause off-target effects, and safe delivery of the autologous cells and tissues for assorted studies and imaging. This individualized iPSCs genomics platform, to evaluate the drug safety in vitro, provides a strong foundation that reduces the risk of evaluation in vivo which will reduce cost in the long run and save animal lives.

### EXPERIMENTAL RESULTS

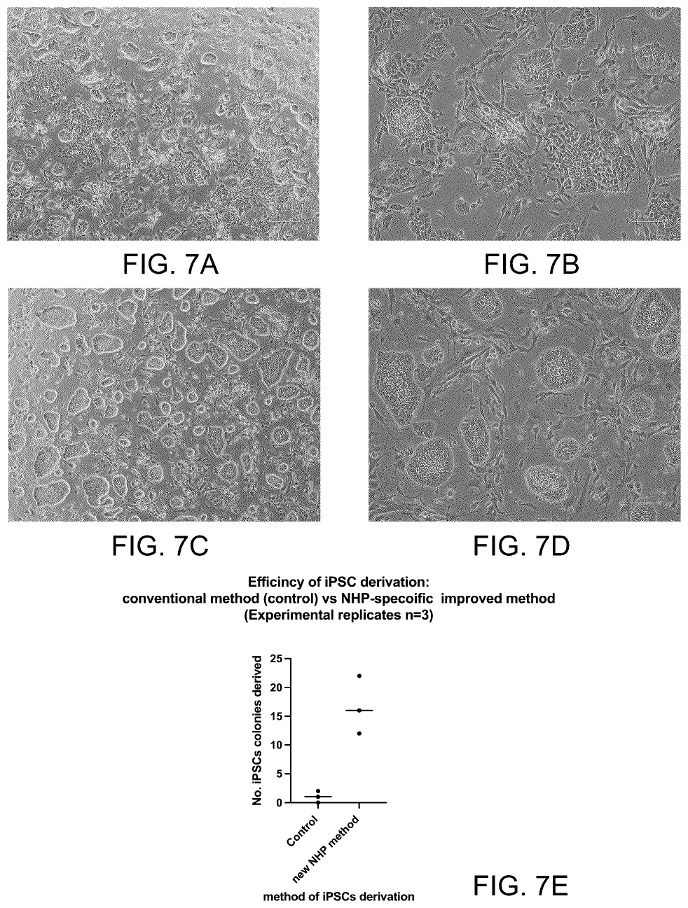

FIGS. 7A-7B are brightfield microscopy images (4× and 10×, respectively) of macaque iPSCs derived using conventional primate iPSCs culture condition, according to the protocols in Takahashi et al. “Induction of Pluripotent Stem Cells from Adult Human Fibroblasts by Defined Factors.” Cell 131, 861-782, Nov. 30, 2007, which is herein incorporated by reference in its entirety.

In brief, the conventional methods and compositions included: iPSCs were generated and maintained in Primate ES medium (ReproCELL, Japan) supplemented with 4 ng/ml recombinant human basic fibroblast growth factor (bFGF, WAKO, Japan). For passaging, human iPSCs were washed once with PBS and then incubated with DMEM/F12 containing 1 mg/ml collagenase IV (Invitrogen) at 37 degrees Celsius. When colonies at the edge of the dish started dissociating from the bottom, DMEF/F12/collagenase was removed and washed with Primate ES cell medium. Cells were scraped and an appropriate volume of the medium was added, and the contents were transferred to a new dish on SNL feeder cells. The split ratio was routinely 1:3. The cells were transfected with pMXs vectors with Fugene 6 transfection reagent (Roche). Twenty-four hours after transfection, the medium was collected as the first virus-containing supernatant and replaced with a new medium, which was collected after twenty-four hours as the second virus-containing supernatant. Human fibroblasts expressing mouse Slc7a1 gene were seeded one day before transduction. The virus-containing supernatants were filtered and supplemented with 4 mg/ml polybrene. Equal amounts of supernatants containing each of the four retroviruses were mixed, transferred to the fibroblast dish, and incubated overnight. Twenty-four hours after transduction, the virus-containing medium was replaced with the second supernatant. Six days after transduction, fibroblasts were harvested by trypsinization and replated on an SNL feeder layer. Next day, the medium was replaced with Primate ES cell medium supplemented with 4 ng/ml bFGF. The medium was changed every other day. Thirty days after transduction, colonies were picked up and transferred into 0.2 ml of Primate ES cell medium. The colonies were mechanically dissociated to small clamps by pipeting up and down. The cell suspension was transferred on SNL feeder in 24-well plates. As shown in FIGS. 7A-7B, in the conventional culture condition, a large proportion of macaque iPSCs spontaneously differentiate as observed in the microscopy images.

In contrast, FIGS. 7C-7D show brightfield microscopy images (4× and 10×, respectively) of macaque iPSCs derived using the compositions and methods described above in the methods and compositions section and as shown in FIGS. 3A-3B. As shown in FIGS. 7C-7D, the iPSCs culture using the improved compositions and methods described herein show iPSCs with round-shape edges, maintaining their pluripotency features in the improved culture conditions.

### EXAMPLES

Example 1. A method for deriving individual pluripotent stem cells (iPSCs) from individuals within a population of nonhuman primates (NHP) comprising: obtaining peripheral blood mononuclear cells (PBMCs) of at least one individual of a population comprising a single species of nonhuman primate; culturing the PBMCs to expand blood progenitor cells in a hematopoietic stem cell (HSC) expansion medium for a predetermined time period; transfecting the cultured PBMCs with a combination of transcription factors so that the cells are induced to overexpress the transcription factors, wherein the transfected cultured PBMCs are reprogrammed into iPSCs; no more than one day post-transfection, transferring the transfected cells into a container comprising a plurality of feeder cells; no more than one day after said transferring, transferring the cells to a second container; on selected days after transfer to the second container, performing at least one of: adding medium to the cells; changing the medium in the cells; and adding one or more cell growth factors to the cells; until a first cell colony appears; passaging the cells by placing each colony in a third container coated with an extracellular matrix; and expanding the first cell colony for use in one or more of: an in vitro test, an in vivo test, or creation of a genome library.
Example 2. The method of any of the preceding examples, wherein the expanding comprises washing the iPSCs with phosphate-buffered saline (PBS) and incubating the iPSCs with a cell detachment solution.
Example 3. The method of any of the preceding examples, wherein the incubating occurs at about 37° C. for a predetermined time period.
Example 4. The method of any of the preceding examples, wherein the expanding comprises aspirating and dissociating the iPSCs into a single cell suspension.
Example 5. The method of any of the preceding examples, further comprising differentiating the iPSCs into main lineages, the main lineages including at least one of: ectoderm, mesoderm, and endoderm.
Example 6. The method of any of the preceding examples, further comprising differentiating the main lineages into final tissues.
Example 7. The method of any of the preceding examples, further comprising generating an individualized genome assembly of the at least one individual.
Example 8. The method of any of the preceding examples, further comprising extracting a quantity of high-molecular weight (HMW) DNA from the obtained PBMCs by means of a process for purification of archival quality DNA.
Example 9. The method of any of the preceding examples, further comprising preparing a genomic library from the extracted HMW DNA; sequencing long-read fragments of HMW DNA; and assembling and annotating the long-read fragments to recreate the genome of the at least one individual from which the PBMCs were obtained.
Example 10. The method of any of the preceding examples, wherein the transcription factors are selected from a group consisting of: c-myc, Klf4, Sox2, Oct3/4, Klf2, Nanog, Tfcp2L1, and Stat3.
Example 11. The method of any of the preceding examples, wherein the plurality of feeder cells are mouse embryonic fibroblast (MEF) cells or SNL feeder cells.
Example 12. The method of any of the preceding examples, wherein the transferring the transfected cells into a container comprising the plurality of feeder cells further comprises adding a tankyrase 1/2 inhibitor in a range of about 1 μM to about 3 μM.
Example 13. The method of any of the preceding examples, wherein a ratio of transfected cells to the plurality of feeder cells is about 1:3 to about 1:7.
Example 14. The method of any of the preceding examples, wherein the transfecting is using a Sendai virus vector.
Example 15. The method of any of the preceding examples, wherein the one or more cell growth factors comprise basic fibroblast growth factor.
Example 16. The method of any of the preceding examples, wherein the passaging the cells by placing each colony in the third container coated with the extracellular matrix comprises adding a serine/threonine kinase inhibitor to a medium in the third container.
Example 17. A method for pre-clinical drug evaluation comprising: an individualized genome assembly for a test subject; and induced pluripotent stem cells (iPSCs) derived from the test subject.
Example 18. The method of Example 17, further comprising implanting cells and tissues derived from iPSCs of the test subject into the test subject to evaluate functionality of the implanted cells and tissues of the test subject.
Example 19. The method of any of Examples 17-18, further comprising generating one or more panels; and predicting whether a particular community of animals is a fit for a drug experiment based on the one or more panels.
Example 20. The method of any of Examples 17-19, further comprising generating a guide RNA or gene-editing tool using the iPSCs derived from the test subject; and performing in vivo testing of the guide RNA or gene-editing tool in the test subject.
Example 21. A method for preclinical testing of human therapies comprising: obtaining tissue or blood from at least one individual of a population comprising a single species of nonhuman primate; isolating PBMCs from the at least one individual; deriving iPSCs from the PBMCs from the at least one individual; extracting DNA from the PBMCs from the at least one individual; preparing a DNA library using, at least in part, the extracted DNA; preparing a full genome assembly for the at least one individual; and performing computational analysis on the full genome assembly.
Example 22. The method of Example 21, wherein the computational analysis comprises identifying one or both of: Major Histocompatibility Complex related genes or Minor Histocompatibility complex related genes.
Example 23. The method of any of Examples 21-22, wherein the computational analysis comprises identifying immune system-related genes.
Example 24. The method of any of Examples 21-23, wherein the computational analysis comprises identifying anti-drug antibody (ADA) response related genes.
Example 25. The method of any of Examples 21-24, wherein the computational analysis comprises performing preclinical genotyping.
Example 26. The method of any of Examples 21-25, wherein the computational analysis comprises identifying liver toxicity related genes.
Example 27. The method of any of Examples 21-26, wherein the DNA is high molecular weight DNA.
Example 28. The method of any of Examples 21-27, wherein the DNA is archival quality DNA.
Example 29. The method of any of Examples 21-28, wherein the preparing the DNA library comprises performing long read sequencing on the extracted DNA.
Example 30. The method of any of Examples 21-29, wherein the preparing the full genome assembly comprises recreating a genome of the at least on individual based on the prepared DNA library.
Example 31. A method for preclinical testing of human therapies comprising: obtaining tissue or blood from at least one individual of a population comprising a single species of nonhuman primate; isolating PBMCs from the at least one individual; deriving iPSCs from the PBMCs from the at least one individual; performing in vitro testing on the derived iPSCs from the at least one individual; deriving in vitro testing results from the in vitro testing; and correlating the in vitro testing results with a genotype of the at least one individual.
Example 32. The method of Example 31, wherein the performing in vitro testing comprises designing a guide RNA and transfecting the iPSCs in vitro with the guide RNA.
Example 33. The method of any of Examples 31-32, wherein the deriving in vitro testing results comprises running a panel to detect a gene editing efficiency of the guide RNA in vitro in the iPSCs.
Example 34. A method for preclinical testing of human therapies comprising: obtaining tissue or blood from at least one individual of a population comprising a single species of nonhuman primate; isolating PBMCs from the at least one individual; deriving iPSCs from the PBMCs from the at least one individual; performing in vivo testing in the at least one individual; deriving in vivo testing results from the in vivo testing; and correlating in vivo testing results from the in vivo testing with a genotype of at least one individual.
Example 35. The method of Example 34, wherein the deriving the iPSCs comprises: culturing the PBMCs to expand blood progenitor cells in a hematopoietic stem cell (HSC) expansion medium for a predetermined time period; transfecting the cultured PBMCs with a combination of transcription factors so that the cells are induced to overexpress the transcription factors, wherein the transfected cultured cells are reprogrammed into iPSCs; no more than one day post-transfection, transferring the transfected cells into a container comprising a plurality of feeder cells; no more than one day after said transferring, transferring the cells to a second container; on selected days after transfer to the second container, performing at least one of: adding medium to the cells; changing the medium in the cells; and adding one or more cell growth factors to the cells; until a first cell colony appears; passaging the cells by placing each colony in a third container coated with an extracellular matrix; and expanding the first cell colony for use in one or more of: an in vitro test, an in vivo test, or creation of a genome library.
Example 36. The method of any of Examples 34-35, wherein the performing in vivo testing comprises autologously transplanting or injecting a subset of the iPSCs into the at least one individual.
Example 37. The method of any of Examples 34-36, further comprising labeling the iPSCs before autologous transplantation or injection.
Example 38. The method of any of Examples 34-37, wherein labeling comprises fluorescent labeling.
Example 39. The method of any of Examples 34-38, wherein the deriving the in vivo testing results comprises running a panel to detect anti-drug antibodies.
Example 40. The method of any of Examples 34-39, wherein the deriving the in vivo testing results comprises running a panel to detect liver toxicity.
Example 41. A method for preclinical testing of human therapies comprising: obtaining tissue or blood from at least one individual of a population comprising a single species of nonhuman primate; isolating PBMCs from the at least one individual; deriving iPSCs from the PBMCs from the at least one individual; performing in vitro testing on a subset of the derived iPSCs from the at least one individual; deriving in vitro testing results from the in vitro testing; autologously injecting or transplanting a second subset of the derived iPSCs from the at least one individual into the at least one individual; performing in vivo testing in the at least one individual; deriving in vivo testing results from the in vivo testing; and correlating the in vivo testing results with the in vitro testing results.
Example 42. The method of Example 41, wherein the deriving iPSCs comprises: culturing the PBMCs to expand blood progenitor cells in a hematopoietic stem cell (HSC) expansion medium for a predetermined time period; transfecting the cultured PBMCs with a combination of transcription factors so that the cells are induced to overexpress the transcription factors, wherein the transfected cultured cells are reprogrammed into iPSCs; no more than one day post-transfection, transferring the transfected cells into a container comprising a plurality of feeder cells; no more than one day after said transferring, transferring the cells to a second container; on selected days after transfer to the second container, performing at least one of: adding medium to the cells; changing the medium in the cells; and adding one or more cell growth factors to the cells; until a first cell colony appears; passaging the cells by placing each colony in a third container coated with an extracellular matrix; and expanding the first cell colony for use in one or more of: an in vitro test, an in vivo test, or creation of a genome library.
Example 43. The method of any of Examples 41-42, wherein the performing in vitro testing comprises designing a guide RNA and transfecting the subset of iPSCs in vitro with the guide RNA.
Example 44. The method of any of Examples 41-43, wherein the deriving in vitro testing results comprises running a panel to detect an in vitro gene editing efficiency of the guide RNA in the subset of iPSCs.
Example 45. The method of any of Examples 41-44, further comprising transfecting iPSCs with the guide RNA for the autologous transplantation or injection.
Example 46. The method of any of Examples 41-45, wherein the deriving the in vivo testing results comprises running a panel to detect liver toxicity.
Example 47. The method of any of Examples 41-46, wherein the deriving the in vivo testing results comprises running a panel to detect an in vivo gene editing efficiency of the guide RNA in the second subset of iPSCs.
Example 48. The method of any of Examples 41-47, wherein the correlating comprises comparing the in vitro gene editing efficiency to the in vivo gene editing efficiency.
Example 49. The method of any of Examples 41-48, wherein the performing in vitro testing comprises treating the iPSCs in vitro with a therapy.
Example 50. The method of any of Examples 41-49, wherein the deriving the in vitro testing results comprises running a panel to detect an in vitro toxicity of the therapy.
Example 51. The method of any of Examples 41-50, wherein the performing the in vivo testing results comprises treating the at least one individual with the therapy.
Example 52. The method of any of Examples 41-51, wherein the deriving the in vivo testing results comprises running a panel to detect an in vivo toxicity of the therapy.
Example 53. The method of any of Examples 41-52, wherein the correlating comprises comparing the in vitro toxicity to the in vivo toxicity of the therapy.

While the foregoing written description of the embodiments enables one of ordinary skill to use the methods described, those of ordinary skill will understand and appreciate the existence of variations, combinations, and equivalents of the specific embodiments, methods, and examples herein. The specification should therefore not be limited by the above-described embodiments, method, and examples, but by all embodiments and methods within the scope and spirit of the attached claims.

As used herein in the description and the claims, a day or one day may be used. A day or one may include about 24 hours+/−about 6 hours, for example.

As used in the description and claims, the singular form “a”, “an” and “the” include both singular and plural references unless the context clearly dictates otherwise. For example, the term “nonhuman primate” may include, and is contemplated to include, a plurality of nonhuman primates. At times, the claims and disclosure may include terms such as “a plurality,” “one or more,” or “at least one”; however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for deriving individual pluripotent stem cells (iPSCs) from individuals within a population of nonhuman primates (NHP) comprising:
obtaining peripheral blood mononuclear cells (PBMCs) of at least one individual of a population comprising a single species of nonhuman primate;
culturing the PBMCs to expand blood progenitor cells in a hematopoietic stem cell (HSC) expansion medium for a predetermined time period;
transfecting the cultured PBMCs with a combination of transcription factors so that the cells are induced to overexpress the transcription factors, wherein the transfected cultured PBMCs are reprogrammed into iPSCs;
no more than one day post-transfection, transferring the transfected cells into a container comprising a plurality of feeder cells;
no more than one day after said transferring, transferring the cells to a second container;
on selected days after transfer to the second container, performing at least one of:
adding medium to the cells;
changing the medium in the cells; and
adding one or more cell growth factors to the cells;
until a first cell colony appears;
passaging the cells by placing each colony in a third container coated with an extracellular matrix; and
expanding the first cell colony for use in one or more of: an in vitro test, an in vivo test, or creation of a genome library.

2. The method of claim 1, wherein the expanding comprises washing the iPSCs with phosphate-buffered saline (PBS) and incubating the iPSCs with a cell detachment solution.

3. The method of claim 2, wherein the incubating occurs at about 37° C. for a predetermined time period.

4. The method of claim 2, wherein the expanding comprises aspirating and dissociating the iPSCs into a single cell suspension.

5. The method of claim 1, further comprising differentiating the iPSCs into main lineages, the main lineages including at least one of: ectoderm, mesoderm, and endoderm.

6. The method of claim 5, further comprising differentiating the main lineages into final tissues.

7. The method of claim 1, further comprising generating an individualized genome assembly of the at least one individual.

8. The method of claim 7, further comprising extracting a quantity of high-molecular weight (HMW) DNA from the obtained PBMCs by means of a process for purification of archival quality DNA.

9. The method of claim 8, further comprising preparing a genomic library from the extracted HMW DNA; sequencing long-read fragments of HMW DNA; and assembling and annotating the long-read fragments to recreate the genome of the at least one individual from which the PBMCs were obtained.

10. The method of claim 1, wherein the transcription factors are selected from a group consisting of: c-myc, Klf4, Sox2, Oct3/4, Klf2, Nanog, Tfcp2L1, and Stat3.

11. The method of claim 1, wherein the plurality of feeder cells are mouse embryonic fibroblast (MEF) cells or SNL feeder cells.

12. The method of claim 1, wherein the transferring the transfected cells into a container comprising the plurality of feeder cells further comprises adding a tankyrase 1/2 inhibitor in a range of about 1 μM to about 3 μM.

13. The method of claim 1, wherein a ratio of transfected cells to the plurality of feeder cells is about 1:3 to about 1:7.

14. The method of claim 1, wherein the transfecting is using a Sendai virus vector.

15. The method of claim 1, wherein the one or more cell growth factors comprise basic fibroblast growth factor.

16. The method of claim 1, wherein the passaging the cells by placing each colony in the third container coated with the extracellular matrix comprises adding a serine/threonine kinase inhibitor to a medium in the third container.

17.-53. (canceled)
